(19) United States
(12) Patent Application Publication
(10) Pub. No.: US 2022/0023532 A1
(43) Pub. Date: Jan. 27, 2022

(54) DEVICE FOR PREVENTING ACCIDENTAL SEPARATION OF A DRIP TUBE FROM AN IV MEDICATION BAG OR BOTTLE

(71) Applicant: Tracy A. Lannan, Derry, NH (US)

(72) Inventor: Tracy A. Lannan, Derry, NH (US)

(21) Appl. No.: 16/939,314

(22) Filed: Jul. 27, 2020

(51) Int. Cl. A61M 5/14

(52) U.S. Cl. CPC A61M 5/1418; A61M 5/1411

(57) ABSTRACT

Apparatus for preventing accidental separation of an IV tube or spike from an IV medication bag or bottle, thereby preventing spills or leakage of hazardous medication and/or biohazards. The apparatus includes a top clamp to attach around the stem of a medication bag or around the neck of a bottle, a bottom clamp to attach around a drip chamber of an IV tube, and a connecting portion connected to both the top clamp and the bottom clamp, the connecting portion ensuring that the top clamp cannot be pulled away from the bottom clamp. The top clamp can be removably attachable so as to enable clamping and unclam ping at the bag stem or bottle neck for situations requiring sequential bag changes, in the case of multiple blood products, or multiple IV bottles. Or, the top clamp can be permanently attachable, which is useful in the context of chemotherapy or immune therapy.

### FIELD OF THE INVENTION

This invention relates generally to intravenous (IV) administration equipment, and more particularly to IV administration safety equipment.

### BACKGROUND OF THE INVENTION

Although many safeguards currently exist for safe handling and administration of hazardous medications and blood products, there is still a significant problem that has not yet been addressed. There are still many instances of nurses, patients, hospital staff, and others being accidentally exposed to hazardous liquids, such as cytotoxic chemotherapy medications, immune modulating substances, and blood products, due to an inadvertent or accidental separation of an intravenous medication tubing from the medication/blood product container, such as a bottle or a bag.

For example, when an IV tube spike is pulled out of the neck of a medication bottle, the liquid in the medication bottle leaks out the hole left by the spike. As a result, inadvertently dislodging the IV tubing spike from a medication/blood product bottle results in uncontrolled flow of possibly hazardous liquid from the bottle into the environment. Likewise, when an IV drip tube is pulled away and disconnects from the stem tube of a medication/blood product bag, a spill of the possibly hazardous liquid occurs.

Such incidents can occur when an unsteady or frail patient becomes lightheaded or dizzy, and then grabs the IV tube for support, accidentally dislodging and disconnecting the spike or tubing from a bottle or a bag containing a dangerous or toxic substance (e.g. a cytotoxic chemotherapeutic agent, or blood), thereby spilling the contents of the bottle or bag onto the patient, the furniture, the floor, the IV pump, and/or soaking the patient's clothing. Thus, there still exists a risk of exposure to various hazardous liquids contained in IV bottles and IV bags that various medical personnel work within their daily duties.

### SUMMARY OF THE INVENTION

The invention is a device to prevent the accidental separation of an IV tube or IV spike from an IV medication bag or bottle, thereby preventing spills or leakage of hazardous medication and/or biohazards. The invention can be used with different brands of medication bags and bottles currently in use for containing and administering intravenous infusions.

An embodiment of the invention includes a removably attachable top clamp that enables clamping and unclamping at the medication bag stem or medication bottle neck for situations requiring sequential bag or bottle changes, e.g., multiple blood products, or multiple IV bottles.

Another embodiment of the invention includes a permanently attachable top clamp with no intention of release once clamped, which is useful in cases where chemotherapy or immune therapy is used.

A general aspect of the invention is an apparatus for preventing accidental separation of an intravenous (IV) tube or spike from an IV medication bag or bottle. The apparatus includes: a top clamp configured to attach around a stem of a medication bag or bottle; a bottom clamp configured to attach around a drip chamber of an IV tube; and a connecting portion connected to both the top clamp and the bottom clamp, the connecting portion configurable to ensure that the top clamp cannot be pulled away from the bottom clamp.

In some embodiments, the connecting portion is configured to be length-adjustable.

In some embodiments, the connecting portion includes a length adjustor configured to accommodate a range of stem lengths of various medication bags and bottles.

In some embodiments, the top clamp is a zip tie.

In some embodiments, the top clamp is configured to be permanently attached.

In some embodiments, the top clamp is configured to be removably attached.

In some embodiments, the bottom clamp is a zip tie.

In some embodiments, the bottom clamp is configured to be permanently attached.

In some embodiments, the bottom clamp is configured to be removably attached.

In some embodiments, the bottom clamp and the connecting portion are each configured so as to maintain visibility of fluid drips within the drip chamber of the IV tube.

Another general aspect of the invention is an apparatus for preventing accidental separation of an intravenous (IV) tube or spike from an IV medication bag or bottle. This apparatus includes: a top clamp portion configured to attach around a stem portion of a medication bag or bottle; a bottom clamp portion configured to attach around a drip chamber of an IV tube; and a connecting portion, integral with both the top clamp portion and the bottom clamp portion, the connecting portion configurable to ensure that the top clamp portion cannot be pulled away from the bottom clamp portion.

In some embodiments, the top clamp portion includes a ratchet head end and a tail end.

In some embodiments, the top clamp portion is configured to be permanently attached.

In some embodiments, the top clamp portion is configured to be removably attached.

In some embodiments, the bottom clamp portion includes a ratchet head end and a tail end.

In some embodiments, the bottom clamp portion is configured to be permanently attached.

In some embodiments, the bottom clamp portion is configured to be removably attached.

In some embodiments, the bottom clamp portion and the connecting portion are each configured so as to maintain visibility of fluid drips within the drip chamber of the IV tube.

### BRIEF DESCRIPTION OF THE DRAWINGS

Many additional features and advantages will become apparent to those skilled in the art upon reading the following description, when considered in conjunction with the accompanying drawings, wherein:

FIG. 1 is a front view of an embodiment of the apparatus for attaching an intravenous (IV) tube to an IV medication bag or bottle, the apparatus including a connecting portion that is integral with both the top clamp portion and the bottom clamp portion, the connecting portion including a length adjustment device to make a taut connection between the top clamp portion and the bottom clamp portion.

FIG. 2 is a side view of the embodiment of the apparatus of FIG. 1, shown attached to the neck of an IV medication bottle and to the drip chamber of an IV tube, also showing how the length adjustment device has been adjusted so as to make the connecting portion taut.

FIG. 3 is a front view of an embodiment including a connecting portion that is attached with a rivet or plastic spot weld to both a top clamp and a bottom clamp, the connecting portion including a flat ratchet head, and the top and bottom clamps being zip ties each having a perpendicular ratchet head.

FIG. 4 is a front view of the embodiment of FIG. 3, shown before being attached to the descending tube of an IV medication bag and to the drip chamber of an IV tube.

### DETAILED DESCRIPTION

A zip tie (also called a cable tie) is a fastener that is commonly used to secure groups of wires or cables, for example. A zip tie includes a flexible nylon tape section having teeth on one side that engage with a pawl in a perpendicular ratchet head at the end of the nylon tape section. As the nylon tape section is pulled through the head, the zip tie tightens and cannot come undone because the ratchet head permits only one-way passage of the teeth through the ratchet head. Some ties include a release tab that can be pressed so as to release the ratchet so that the zip tie can be loosened, and then removed, and possibly reused.

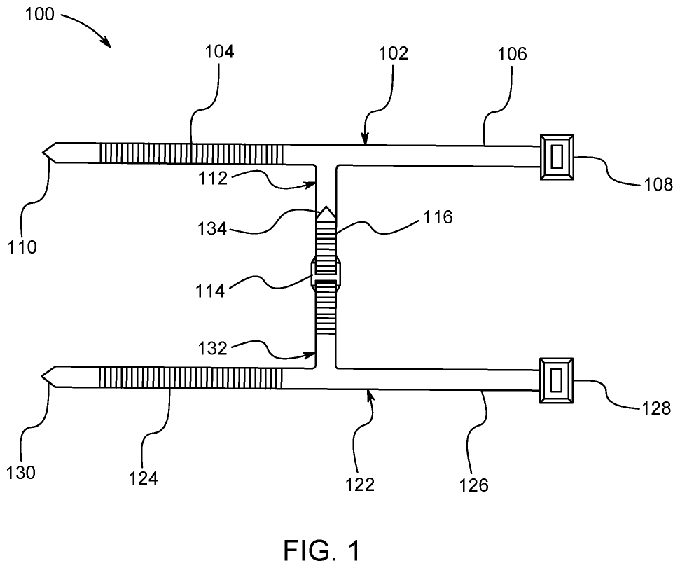

With reference to FIG. 1, an embodiment 100 of the apparatus for attaching an intravenous (IV) tube to an IV medication bag or IV bottle is shown. This embodiment 100 includes a top clamp portion 102 made from flexible nylon tape including a section having teeth 104 on at least one side, a smooth section 106, and a ratchet head 108 having a pawl configured to engage with the teeth 104 after a tail end 110 is inserted perpendicularly into the ratchet head 108 after wrapping the top clamp portion 102 around a IV medication bag or IV bottle.

In some embodiments, the ratchet head 108 includes a release tab to release the teeth 104. In other embodiments, the ratchet head 108 does not include a release tab to release the teeth 104, and so the top clamp portion 102 would remain attached to the IV medication bag or IV bottle until the top clamp portion 102 is cut off.

The top clamp portion 102 also integrally includes a first perpendicular section 112 without teeth that terminates in a low-profile (flat) ratchet head 114. The flat ratchet head 114 is configured to receive in parallel relationship a second perpendicular section 132 with teeth 116 that terminates in a tail end 134. A pawl in the flat ratchet head 114 is configured to engage with the teeth 116 of the second perpendicular section 132 such that the second perpendicular section 132 can be inserted into the flat ratchet head 114 to an extent selected by the user, and cannot be withdrawn unless a release tab of the flat ratchet head 114 is pressed by the user.

The first perpendicular section 112 and the second perpendicular section 132 together form a connecting portion that is integral with both the top clamp portion 102 and a bottom clamp portion 122. The bottom clamp portion 122 includes the second perpendicular section 132. The second perpendicular section 132 cannot be withdrawn from the top clamp portion 102 unless a release tab of the flat ratchet head 114 is pressed by the user. Thus, the connecting portion can be configured to ensure that the top clamp portion 102 cannot be pulled away from the bottom clamp portion 122.

Accordingly, the embodiment 100 also includes the bottom clamp portion 122 having the second perpendicular section 132. The bottom clamp portion 122 includes a flexible nylon tape including a section having teeth 124 on at least one side, a smooth section 126, and a ratchet head 128 having a pawl configured to engage with the teeth 124 after the tail end 130 is inserted perpendicularly into the ratchet head 128 at the end of the smooth section 126, after wrapping the bottom clamp portion 122 around an intravenous (IV) tube.

In some embodiments, the ratchet head 128 includes a release tab to release the teeth 124. In other embodiments, the ratchet head 128 does not include a release tab to release the teeth 124, and so the bottom clamp portion 122 would remain attached to the intravenous (IV) tube until the bottom clamp portion 122 is cut off.

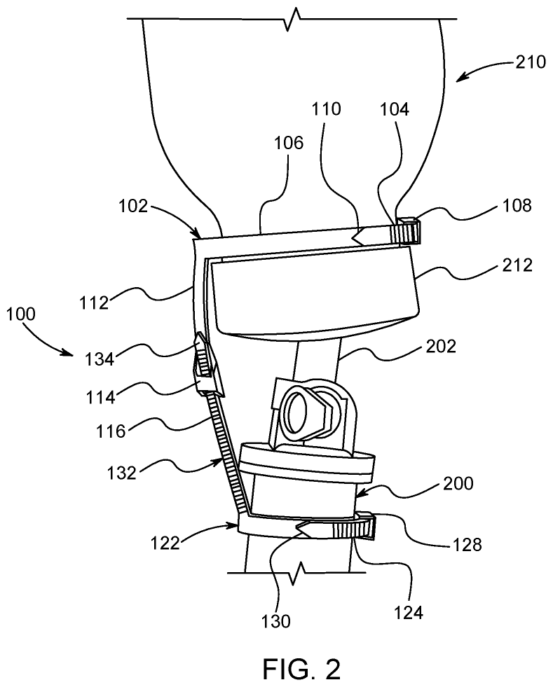

As will be shown in FIG. 2, after wrapping and securing the top clamp portion 102 around an IV medication bag or IV bottle, and after wrapping and securing the bottom clamp portion 122 around an intravenous (IV) tube, the user can pull the bottom clamp portion 122 towards the top clamp portion 102 by pulling the teeth 116 of the second perpendicular section 132 through the flat ratchet head 114 by the tail end 134. Or, the user can pull the teeth 116 of the second perpendicular section 132 through the flat ratchet head 114 by the tail end 134 just to remove slack in the connecting portion.

Referring to FIG. 2, the embodiment 100 of FIG. 1 is shown for attaching an IV drip chamber 200 at a substantially fixed distance from the neck 212 of an IV bottle 210 having a spike 202 extending upward into the IV bottle 210. The drip chamber 200 also has a connected intravenous (IV) tube (not shown) hanging downward. The IV tube can be accidentally pulled or yanked downward, thereby pulling the drip chamber 200 and the spike 202 away from the IV bottle 210.

Without the embodiment 100 attached to both the IV drip chamber 200 and to the neck of the IV bottle 210 , the spike 202 would pull out of the membrane of the neck 212 of the IV bottle 210 when the connected IV tube is pulled downward, causing the contents of the IV bottle 210 to spill out, and causing any liquid in the spike to also spill out.

However, when the top clamp portion 102 is attached around the neck 212 of the IV bottle 210, the bottom clamp portion 122 is attached around the IV drip chamber 200, and the teeth 116 of the second perpendicular section 132 are pulled through the flat ratchet head 114 until the connecting portion is taught, the spike 202 will not pull out of the membrane of the neck 212 of the IV bottle 210 when the connected IV tube is pulled downward, either accidentally or intentionally.

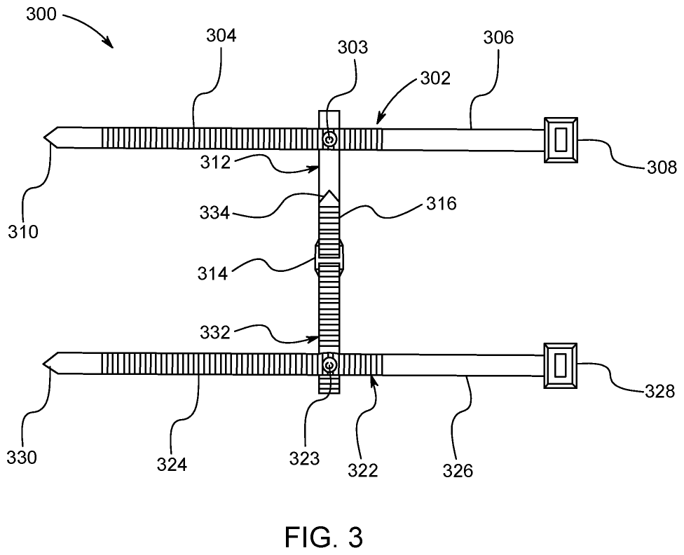

Referring to FIG. 3, another embodiment 300 of the apparatus for securing an intravenous (IV) tube to an IV medication bag or IV bottle is shown. This embodiment 300 includes a top clamp portion 302 made from flexible nylon tape including a section having teeth 304 on at least one side, a smooth section 306, and a ratchet head 308 having a pawl configured to engage with the teeth 304 after a tail end 310 is inserted perpendicularly into the ratchet head 308 after wrapping the top clamp portion 302 around a neck of an IV medication bag or IV bottle.

In some embodiments, the ratchet head 308 includes a release tab to release the teeth 304. In other embodiments, the ratchet head 308 does not include a release tab to release the teeth 304, and so the top clamp portion 302 would remain attached to the IV medication bag or IV bottle until the top clamp portion 302 is cut off.

The top clamp portion 302 also is attached via a fastener 303 (e.g., a plastic rivet, or a plastic spot weld) to a first perpendicular section 312 without teeth that terminates in a low-profile (flat) ratchet head 314. The flat ratchet head 314 is configured to receive in parallel relationship a second perpendicular section 332 with teeth 316 that terminates in a tail end 334. A pawl in the flat ratchet head 314 is configured to engage with the teeth 316 of the second perpendicular section 332 such that the second perpendicular section 332 can be inserted into the flat ratchet head 314 to an extent selected by the user, and cannot be withdrawn unless a release tab of the flat ratchet head 314 is pressed by the user.

The first perpendicular section 312 and the second perpendicular section 332 together form a connecting portion that is fastened to both the top clamp portion 302 and a bottom clamp portion 322 via the fasteners 303 and 323 respectively. The bottom clamp portion 322 has the second perpendicular section 332 attached thereto via the fastener 323. The second perpendicular section 332 cannot be withdrawn from the top clamp portion 302 unless a release tab of the flat ratchet head 314 is pressed by the user. Thus, the connecting portion can be configured to ensure that the top clamp portion 302 cannot be pulled away from the bottom clamp portion 322.

Accordingly, the embodiment 300 also includes the bottom clamp portion 322 having the second perpendicular section 332 attached thereto via the fastener 323. The bottom clamp portion 322 includes a flexible nylon tape including a section having teeth 324 on at least one side, a smooth section 326, and a ratchet head 328 having a pawl configured to engage with the teeth 324 after the tail end 330 is inserted perpendicularly into the ratchet head 328 at the end of the smooth section 326, after wrapping the bottom clamp portion 322 around the drip chamber of an intravenous (IV) tube.

In some embodiments, the ratchet head 328 includes a release tab to release the teeth 324. In other embodiments, the ratchet head 328 does not include a release tab to release the teeth 324, and so the bottom clamp portion 322 would remain attached to the drip chamber of the intravenous (IV) tube until the bottom clamp portion 322 is cut off.

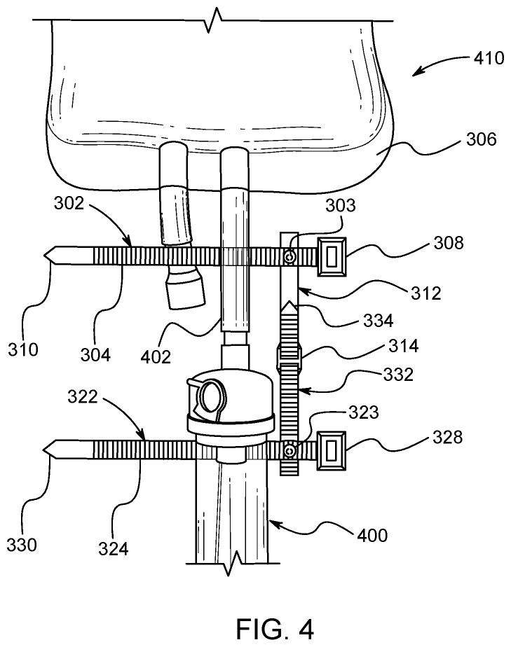

As will be shown in FIG. 4, after wrapping and securing the top clamp portion 302 around a descending tube (“stem”) of an IV medication bag, and after wrapping and securing the bottom clamp portion 322 around the drip chamber 400 of an intravenous (IV) tube, the user can pull the bottom clamp portion 322 towards the top clamp portion 302 by pulling the teeth 316 of the second perpendicular section 332 through the flat ratchet head 314 by the tail end 334. Or, the user can pull the teeth 316 of the second perpendicular section 332 through the flat ratchet head 314 by the tail end 334 to remove slack in the connecting portion.

Referring to FIG. 4, the embodiment 300 of FIG. 3 is shown in an open condition before the top clamp portion 302 has been secured around the descending tube (“stem”) 402 of an IV medication bag 410, and before the bottom clamp portion 322 has been secured around the drip chamber 400 of the intravenous (IV) tube. After the top clamp portion 302 and the bottom clamp portion 322 have been secured, the user can push and then pull the tail end 334 of the second perpendicular section 332 through the flat ratchet head 314 until any slack in the connecting portion is removed.

Now, the top clamp portion 302 cannot be pulled away from the bottom clamp portion 322, and consequently pulling on the drip chamber 400, or any tube connected to the drip chamber, will not result in the drip chamber 400 disconnecting from the descending tube (“stem”) 402 of the IV medication bag 410, thereby preventing the liquid contents of the medication bag from creating a hazardous spill by unintentionally pouring out of the descending tube (“stem”) 402.

The embodiment 300 can be made from two standard zip ties and a single flat zip tie, which are presently commercially available. A person of average skill in the art could use the flat zip tie as the connecting portion by cutting the flat zip tie in half, and fastening the cut end of each half using a rivet or a spot weld along each of the top and bottom clamps, respectively, at a point along each clamp suitable to fit the circumference of the descending tube (“stem”) 402 and the drip chamber 400, respectively. The flat zip tie connector cut in half and attached in this way provides adjustable separation between the two standard zip ties.

Other modifications and implementations will occur to those skilled in the art without departing from the spirit and the scope of the invention as claimed. Accordingly, the above description is not intended to limit the invention, except as indicated in the following claims.

What is claimed is:

1. Apparatus for preventing accidental separation of an intravenous (IV) tube or spike from an IV medication bag or bottle, the apparatus comprising:
a top clamp configured to attach around a stem of a medication bag or bottle;
a bottom clamp configured to attach around a drip chamber of an IV tube; and
a connecting member connected to both the top clamp and the bottom clamp, the connecting member configurable to ensure that the top clamp cannot be pulled away from the bottom clamp.

2. The apparatus of claim 1, wherein the connecting portion is configured to be length-adjustable.

3. The apparatus of claim 1, wherein the connecting portion includes a length adjustor configured to accommodate a range of stem lengths of various medication bags and bottles.

4. The apparatus of claim 1, wherein the top clamp is a zip tie.

5. The apparatus of claim 1, wherein the top clamp is configured to be permanently attached.

6. The apparatus of claim 1, wherein the top clamp is configured to be removably attached.

7. The apparatus of claim 1, wherein the bottom clamp is a zip tie.

8. The apparatus of claim 1, wherein the bottom clamp is configured to be permanently attached.

9. The apparatus of claim 1, wherein the bottom clamp is configured to be removably attached.

10. The apparatus of claim 1, wherein the bottom clamp and the connecting portion are each configured so as to maintain visibility of fluid drips within the drip chamber of the IV tube.

11. Apparatus for preventing accidental separation of an intravenous (IV) tube or spike from an IV medication bag or bottle, the apparatus comprising:
a top clamp portion configured to attach around a stem of a medication bag or a neck of a bottle;
a bottom clamp portion configured to attach around a drip chamber of an IV tube; and
a connecting portion, integral with both the top clamp portion and the bottom clamp portion, the connecting portion configurable to ensure that the top clamp portion cannot be pulled away from the bottom clamp portion.

12. The apparatus of claim 11, wherein the top clamp portion includes a ratchet head end and a tail end.

13. The apparatus of claim 11, wherein the top clamp portion is configured to be permanently attached.

14. The apparatus of claim 11, wherein the top clamp portion is configured to be removably attached.

15. The apparatus of claim 11, wherein the bottom clamp portion includes a ratchet head end and a tail end.

16. The apparatus of claim 11, wherein the bottom clamp portion is configured to be permanently attached.

17. The apparatus of claim 11, wherein the bottom clamp portion is configured to be removably attached.

18. The apparatus of claim 11, wherein the bottom clamp portion and the connecting portion are each configured so as to maintain visibility of fluid drips within the drip chamber of the IV tube.
